(19) United States
(12) Patent Application Publication
(10) Pub. No.: US 2010/0055188 A1
(43) Pub. Date: Mar. 4, 2010

(54) NANO-VALVES FOR SMALL-MOLECULE DRUG DELIVERY

(76) Inventor: Dong June Ahn, Seoul (KR)

Correspondence Address: EDWARDS ANGELL PALMER & DODGE LLP, P.O. BOX 55874, BOSTON, MA, 02205, US

(21) Appl. No.: 12/200,877

(22) Filed: Aug. 28, 2008

(51) Int. Cl. A61K 9/14

(52) U.S. Cl. 424/489; 977/773

(57) ABSTRACT

A system for drug delivery including a plurality of molecular-valves that are responsive to an exterior stimulus so as to selectively open in response to the stimulus. A quantity of a drug is initially contained within the molecular-valves. The molecular-valves are associated with the surface (e.g., both the exterior surface, as well as within the internal pore structure) of the substrate. Upon exposure to a selected stimulus, the molecular-valves open, resulting in release of the drug molecules.

### TECHNICAL FIELD

The present application relates generally to nanotechnology methods and structure for use in drug delivery.

### BACKGROUND

Drugs are used in the practice of medicine to treat a wide variety of conditions. Currently, drug delivery is typically accomplished either by oral ingestion or intravenously. Oral ingestion methods are more commonly used by patients in a home setting as they provide for more convenient administration of a desired drug. Although administration of a dosage of a drug is conveniently achieved in this manner, it is often difficult to control release of the drug from the substrate carrier (e.g., in the form of a pill) which is ingested. For example, sometimes portions of the drug may remain with the substrate carrier, the result being that they are not released as desired, but instead simply pass through the digestive system of the patient. In addition, current drug delivery systems often do not adequately provide for long-lasting release of a desired drug dosage, but may require repeat dosages at regular intervals in an attempt to maintain a desired concentration of the drug within the patient's system.

### BRIEF SUMMARY

According to one embodiment, a system for drug delivery is provided. Such a system includes a plurality of molecular-valves that are responsive to an exterior stimulus so as to selectively open in response to the stimulus. A quantity of a drug is initially contained within the molecular-valves (e.g., a small number of molecules of a desired drug are initially contained within the molecular-valves, depending on the size of the storage chamber defined by the molecular-valve and the relative size of the drug molecule). The molecular-valves are associated with (e.g., located on) the surface of a porous substrate (e.g., a micro-particle). The valves may be associated with the exterior surface of the substrate, as well as within the internal pore structure of the substrate.

According to one embodiment, the stimulus which causes the molecular-valve to open, permitting release of the quantity of the drug contained within the internal chamber of the molecular-valve comprises one or more of photonic energy, electrical energy, a magnetic field, or a chemical concentration (e.g., which results in a chemical reaction altering the structural shape of the molecular-valve so as to release the drug).

In one embodiment, the drug delivery system may be prepared by providing a plurality of molecular-valves in which each molecular-valve includes a molecular framework defining an interior chamber. In one embodiment, a quantity of a drug may be initially contained within the interior chambers of the molecular-valves. In another embodiment, the quantity of drug may be introduced into the internal chambers. The drug-filled molecular valves are dispersed onto a porous substrate so as to be associated with the surface area of the porous substrate. In one embodiment, the molecular-valves are associated with (e.g., located on) both the external surface area of the substrate, as well as within the internal pores of the porous substrate.

In one method, a selected drug may be delivered by providing a drug delivery system as described above including a plurality of drug-filled molecular-valves associated with the surface area of a porous substrate, and exposing the molecular-valves of the drug delivery system to an exterior stimulus configured to selectively open the molecular-valves so as to release the drug contained therein.

This summary is provided to introduce a selection of concepts in a simplified form that are further described below in the Detailed Description. This summary is not intended to identify key features or essential characteristics of the claimed subject matter, nor is it intended to be used as an aid in determining the scope of the claimed subject matter.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A is a schematic representation of an illustrative molecular-valve in a closed position including a drug molecule initially trapped within the molecular-valve.

FIG. 1B is a schematic representation of the molecular-valve of FIG. 1A, but in an open position which permits the drug molecule to be released.

FIG. 2A is an illustration of an illustrative embodiment of a porous micro-particle including a plurality of molecular-valves containing drug molecules in which the molecular-valves are associated with the exterior surface of the micro-particle as well as within pores of the micro-particle.

FIG. 2B is an illustration of the porous micro-particle of FIG. 2A in which the plurality of molecular-valves are open and the drug molecules are being released.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein.

“Micro particles” are micron-scale particles which can have a high surface area, e.g., as a result of outer surfaces and internal porosity. Micro particles offer surfaces for attachment, immobilization or other association of molecular-valves both on the external surface of the micro particles as well as within the internal pore surfaces. Micro particles permit molecular-valves to be immobilized and therefore employed as a solid material while providing a relatively high concentration or number of molecular-valves within a given volume. By providing a high surface area for attachment of molecular-valves, micro particles can increase the rate of drug delivery per volume of substrate as compared to conventional alternatives.

According to one embodiment, a system for drug delivery is provided. Such a system includes a plurality of molecular-valves that are responsive to an exterior stimulus so as to selectively open in response to the stimulus. A quantity of a drug is initially contained within the molecular-valves. The molecular-valves are associated with the surface of a porous substrate. The valves may be disposed on or otherwise associated with the exterior surface of the substrate, as well as within the internal pore structure of the substrate.

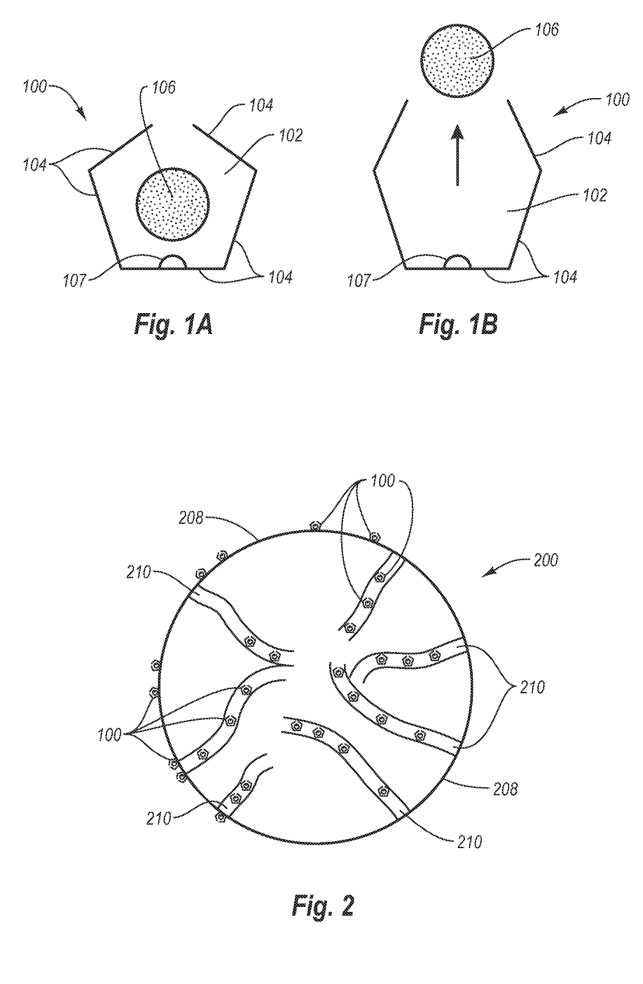

FIG. 1A illustrates a schematic example of a molecular-valve 100. As illustrated in FIG. 1A, molecular-valve 100 is closed so as to define an internal chamber 102. Chamber 102 is bounded by walls 104. Although the schematic illustration shows walls 104 defining a chamber of a particular shape (e.g., substantially pentagonal), the molecular-valve and walls 104 may provide an internal (and/or external) chamber of any shape. Walls 104 of molecular-valve 100 may be formed of a carbon based material, for example an organic carbon backbone structure. The structure may comprise, for example, a cyclophane structure, formed of large molecular rings. In another embodiment, the walls 104 may comprise a protein structure. A drug molecule 106 is initially contained within chamber 102 with the molecular-valve 100 in a closed configuration. As shown, the closed configuration does not necessarily require that the chamber define an entirely bounded, closed space, only that any openings or discontinuities between walls 104 be sufficiently small so as to prevent premature release of drug molecule 106. In other words, drug molecule 106 is trapped within chamber 102 when the molecular-valve is in a closed configuration.

The molecular-valve 100 may include a receptor site 107 to which the drug molecule is attracted or with which the drug molecule 106 is otherwise associated. Such a receptor may provide one illustrative method for filling the molecular-valves during manufacture, as the drug molecules would be attracted to the receptor site within the open molecular-valve. In another embodiment, the molecular valve may be built around the drug molecule. Such an embodiment may not require that the molecular-valve be reversible, capable of both opening and closing repeatedly, as all that would be required once the drug molecule is trapped within the molecular-valve would be a single opening action, releasing the drug molecule.

As shown in FIG. 1B, in response to a stimulus the molecular-valve 100 opens, allowing release of drug molecule 106 from internal chamber 102. Examples of such external stimuli which may result in opening of molecular-valve 100 and release of drug molecule 106 include, but are not limited to, exposure to photonic or other electromagnetic energy (e.g., light of a particular wavelength), electrical energy (e.g., application of a particular voltage and/or current), a magnetic field (e.g., of a particular strength), or a chemical concentration. For example, exposure to a particular concentration of a given chemical may result in a redox chemical reaction which alters the structural shape of the molecular-valve, causing it to open. In a reversible embodiment employing redox chemistry an oxidation reaction may result in the molecular-valve opening, and a reduction reaction may result in reclosing of the internal chamber 102 (or vice versa).

For additional information regarding organic, molecular-valve molecules that can be operated under photonic, electrical, magnetic, or chemical stimuli, their manufacture, use and properties, reference is made to Nguyen, Thoi D. et al., A Reversible Molecular Valve, Proceedings of the National Academy of Sciences of the United States of America Vol. 102 No. 29 (Jul. 19, 2005) pp. 10029-10034, and also Browne, Wesley R. et al. Making Molecular Machines Work, Vol. 1 October 2006, pp. 25-35, the disclosures of which are incorporated herein by reference. For example, the Nguyen article describes a rotaxane cyclic molecule, which is an example of a cyclophane. The rotaxane molecule includes a ring portion that moves from one attachment location to another upon addition of Fe(ClO₄)₃. Such geometric rearrangement of the molecule's structure can be exploited to operate as a molecular-valve. Movement and reattachment of the ring portion to a different location relative to the molecule effected by addition of Fe(ClO₄)₃ can be reversed by addition of a weak acid (e.g., ascorbic acid).

For example, an illustrative molecular-valve may comprise a protein structure including an internal channel surrounded by the protein structure. One such channel protein is described in the Browne article. The channel protein of the Browne article comprises a channel protein modified with a photochemical active spiropyran switch, as illustrated below.

The reversible switch acts as a valve control for a 3 nm channel. The valve can be opened and closed upon exposure to ultraviolet and visible light, respectively. This is possible as the neutral switch spiropyran molecule converts to a highly polar zwitterionic form upon exposure to ultraviolet light. Upon exposure to visible light, the spiropyran molecule, as illustrated above, converts back to its neutral configuration, closing the channel. Such an embodiment is an example of a molecular-valve that is reversible. Other configurations may be capable of opening and/or closing only a single time.

Another example of an illustrative molecular-valve may control movement in and/or out of a channel or internal chamber allosterically. A ligand-gated opening including an azobenzene optical switch may rely on the cis to trans photo-isomerization of azobenzene, with its resulting large geometric change in the molecule, and as a consequence, blocking or unblocking of an opening to the chamber. An example of an azobenzene optical switch is illustrated below:

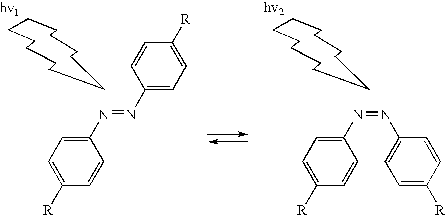

The stimulus in such an example is exposure to a first wavelength of light to convert from a trans to cis configuration, followed by exposure to a second, different wavelength of light to revert back to the original configuration. In the illustration, the R groups may represent any relatively bulky group to which the benzene rings are attached (e.g., an aliphatic, cyclophane, and/or protein portion). In addition, it is not necessary that the R groups be identical. As shown, when in a trans configuration in which the R groups are on different sides of the N═N double bond, the azobenene molecule provides an open configuration in which a drug molecule would be released. When in a cis configuration in which the bulky R groups are on the same side of the N═N double bond, the azobenzene molecule is capable of trapping a drug molecule within the largely triangular space between the R groups and the N═N double bond.

According to one embodiment, the drug molecules may be introduced into the host molecular-valves by soaking of the molecular-valves in an opened configuration within a solution containing the drug compound to be trapped within the molecular-valves. The concentration gradient between the internal chambers (initially zero) and the solution will naturally result in diffusion of the drug molecules towards and into the internal chambers of the molecular-valves. In embodiments where the molecular-valve includes a receptor on the inside wall 104 of the chamber 102 that attracts the target drug molecule(s), filling of the molecular-valves may be accomplished even more efficiently as the drug molecules are actively attracted to the receptor site within internal chamber 102. Depending on the size of the drug molecule and the relative size of internal chamber 102, one or more of the drug molecules may fit within each molecular valve 100. Of course, during manufacture, some fraction of the molecular-valves may remain “empty”, but this fraction can be reduced through manipulation of the soak time when using a concentration gradient and/or through use of receptor sites within the internal chambers, as described above. Once filled, the molecular-valves may be closed (e.g., by exposure to a particular stimulus), trapping the drug molecules within the molecular-valves. The drug molecules may thus be stored within the molecular-valves until it is desired to cause their release (e.g., within a patient).

In another example, the molecular-valves may be built around the drug molecules. Such an embodiment may not require that the molecular valve be reversible, capable of both opening and closing repeatedly. All that would be required once the drug molecule is trapped within the molecular-valve would be a single opening action, releasing the drug molecule. Of course the stimulus selected to cause opening of the molecular-valve is not applied until it is desired to release the drug molecules. Such an embodiment may also employ a receptor to attract or otherwise associate the drug molecule at a desired location relative to the wall 104 as wall 104 is constructed around one or more of the drug molecules 106.

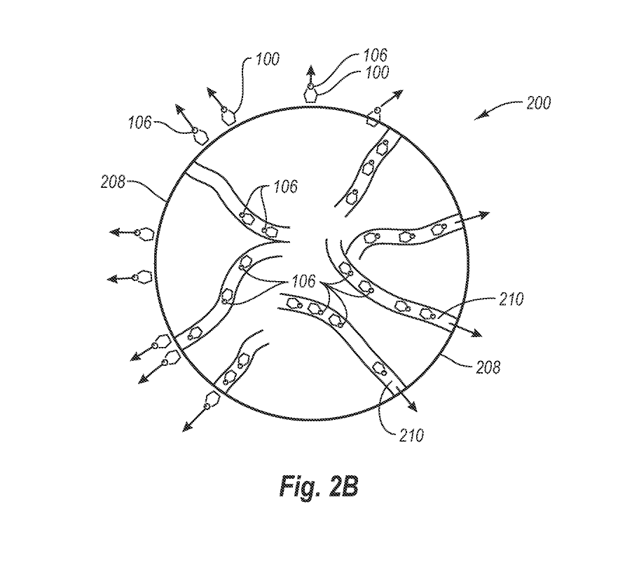

As shown in FIG. 2A, a plurality of the molecular-valves 100 may be embedded within a porous substrate, an example of which is illustrated porous micro-particle 200. As illustrated in FIG. 2A, the molecular-valves 100 may be associated with (e.g., located on) both the external surface area 208 of the porous micro-particle 200, as well as within the internal pores 210 of the porous micro-particle 200. Embedding of the molecular-valves may be accomplished by any desired method. For example, a porous substrate may be soaked or otherwise exposed to a solution, mixture, or quantity of the drug filled molecular-valves (as shown in FIG. 1A). In another embodiment, the molecular-valves may be embedded within the porous substrate prior to filling with drug molecules. Through diffusion and concentration gradient, the molecular-valves diffuse so as to become embedded within and on the porous substrate (e.g., porous micro-particle 200). In one example, the porous substrate may include receptor sites which serve to attract molecular-valves 100, increasing the loading and efficiency with which micro-particle 200 or other porous substrate is embedded with molecular-valves 100. Upon exposure to an exterior stimulus as described above, the molecular-valves open, releasing drug molecules 106, as shown in FIG. 2B.

Any known type of micro particle or other shaped porous substrate can be used to immobilize molecular-valves according to this disclosure. Non-limiting examples of porous substrates include micro particles or other shaped substrates which comprise at least one of glass, silica, latex, polystyrene, carbon, silver, copper, other metal, or magnetic material. According to one embodiment, the micro particles may have a size in a broad range of about 0.1 micron to about 1000 microns, in an intermediate range of about 0.25 micron to about 250 microns, or in a narrow range about 0.5 micron to about 100 microns. According to another embodiment, the porous substrate may be formed of a biocompatible and/or bioresorbable material (e.g., a polyethylene glycol (PEG), polylactic acid (PLA), polyglycolic acid materials (PGA), polylactic-polyglycolic copolymers (PLGA), and combinations thereof).

The porous substrate (e.g., micro-particle) has a specific surface area in a broad range of about 1 m²/kg to about 5,000,000 m²/kg. The porous substrate has a specific surface area in an intermediate range of about 10 m²/kg to about 500,000 m²/kg. The porous substrate has a specific surface area in a narrow range of about 100 m²/kg to about 50,000 m²/kg.

The molecular-valves have a concentration in a broad range of about 1 pg/cm³ to about 1 g/cm³ by weight of the porous substrate embedded with a plurality of molecular-valves. The molecular-valves have a concentration in an intermediate range of about 100 ng/cm³ to about 100 μg/cm³ by weight of the porous substrate embedded with a plurality of molecular-valves. The molecular-valves have a concentration in a narrow range of about 1 ng/cm³ to about 100 μg/cm³ by weight of the porous substrate embedded with a plurality of molecular-valves.

Such a porous substrate embedded with molecular-valves containing drug molecules may be used to deliver a drug orally (e.g., one or more of the chemical concentrations within the patient's stomach or digestive track may serve to open the molecular-valves) or otherwise. By alternative example, the micro-particles may be delivered in an atomized nasal spray in which the molecular-valves comprise azobenzene molecules as described above. Release of the drug molecules in such a delivery mode may be triggered in such an embodiment by exposure of the molecular-valves to light of a first wavelength. Other examples may include similar or alternative delivery modes (e.g., oral, nasal, intravenous, or otherwise) and may be triggered by chemicals present within the nasal or other passages where the drug is delivered, by a secondary application of a chemical stimulus (which may precede or follow application of the drug), by light or electrical stimulus, a magnetic field, or any other conceivable stimulus.

Examples of drug molecules that may be delivered with the system include, but are not limited to, anti-proliferative/antimitotic agents including, but not limited to, natural products such as vinca alkaloids (i.e., vinblastine, vincristine, and vinorelbine), paclitaxel, epidipodophyllotoxins (i.e., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e., estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e., aspirin; para-aminophenol derivatives i.e., acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), everolimus, azathioprine, mycophenolate mofetil); angiogenic agents: vascular endothelial growth factor (VEGF), fibroblast growth factor (FGF); angiotensin receptor blockers; nitric oxide donors; antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors. Also, it should be recognized that many active agents have multiple pharmaceutical uses other than those specifically recited.

Such drugs or beneficial agents can include antithrombotics, anticoagulants, antiplatelet agents, thrombolytics, antiproliferatives, anti-inflammatories, agents that inhibit hyperplasia, inhibitors of smooth muscle proliferation, antibiotics, growth factor inhibitors, or cell adhesion inhibitors, as well as antineoplastics, antimitotics, antifibrins, antioxidants, agents that promote endothelial cell recovery, antiallergic substances, radiopaque agents, viral vectors having beneficial genes, genes, siRNA, antisense compounds, oligionucleotides, cell permeation enhancers, proteins, polypeptides, nucleic acids, polynucleotides, polynucleotide duplexes, siRNA, miRNA, prodrugs, molecular probes, oligopeptides, polypeptides, proteins, oligonucleotides, polynucleotides, DNA, RNA, siRNA, nucleic acids, carbohydrates, or lipids, and combinations of any of the foregoing. Another example of a suitable beneficial agent is described in U.S. Pat. No. 6,015,815 and U.S. Pat. No. 6,329,386 entitled “Tetrazole-containing rapamycin analogs with shortened half-lives”, the entireties of which are herein incorporated by reference.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system for drug delivery comprising:
a plurality of molecular-valves, each molecular-valve including a molecular framework defining an interior chamber, the molecular valves being responsive to exterior stimuli so as to selectively open in response to a stimulus;
a quantity of a drug, the drug being initially contained within the plurality of molecular-valves; and
a porous substrate including a surface area, the plurality of molecular-valves being associated with the surface area of the porous substrate.

2. A system as recited in claim 1, wherein the porous substrate comprises a porous micro-particle.

3. A system as recited in claim 1, wherein at least a portion of the plurality of molecular-valves are disposed within internal pores of the porous substrate.

4. A system as recited in claim 1, wherein at least a portion of the plurality of molecular-valves are disposed on an exterior surface of the porous substrate.

5. A system as in claim 1, wherein the molecular-valves are present in a range of about 1 ng/cm³ to about 100 μg/cm³ of the porous substrate.

6. A system as in claim 1, wherein the porous substrate comprises at least one of glass, silica, latex, polystyrene, carbon, silver, copper, or metal.

7. A system as in claim 1, wherein the porous substrate comprises at least one of a polyethylene glycol, polylactic acid, polyglycolic acid, or a polylactic acid-polyglycolic acid copolymer.

8. A system as in claim 1, wherein the porous substrate has a specific surface area in a range of about 100 m²/kg to about 50,000 m²/kg.

9. A system as recited in claim 1, wherein the plurality of molecular-valves are activated so as to release the drug initially contained therein by one or more stimuli selected from the group consisting of photonic energy, electrical energy, a magnetic field, and a chemical concentration.

10. A system as recited in claim 1, wherein the plurality of molecular-valves are activated by exposure to a chemical concentration which results in a redox chemical reaction which alters the shape of the molecular-valve so as to permit release of the drug initially contained therein.

11. A method for manufacturing a porous substrate embedded with a plurality of molecular-valves comprising:
providing a plurality of molecular-valves, each molecular-valve including a molecular framework defining an interior chamber, a quantity of a drug being initially contained within the interior chambers of the molecular-valves; and
dispersing the molecular-valves onto and/or within a porous substrate.

12. A method as recited in claim 11, wherein the porous substrate comprises a porous micro-particle.

13. A method as in claim 11, wherein the molecular-valves are dispersed onto the porous substrate by means of a solvent.

14. A method as in claim 13, wherein the molecular-valves are in the form of a solution or suspension within the solvent.

15. A method as recited in claim 11, wherein at least a portion of the plurality of molecular-valves are embedded within internal pores of the porous substrate and another portion of the plurality of molecular-valves are disposed on an exterior surface of the porous substrate.

16. A method as in claim 11, wherein the molecular-valves have a concentration in a range of about 1 ng/cm³ to about 100 μg/cm³ of the porous substrate.

17. A method as in claim 11, wherein the porous substrate comprises at least one of a polyethylene glycol, polylactic acid, polyglycolic acid, or a polylactic acid-polyglycolic acid copolymer.

18. A method as in claim 11, wherein the porous substrate has a specific surface area in a range of about 100 m²/kg to about 50,000 m²/kg.

19. A method for drug delivery comprising:
providing a drug delivery system comprised of:
a plurality of molecular-valves, each molecular-valve including a molecular framework defining an interior chamber, the molecular-valves being responsive to exterior stimuli so as to selectively open in response to a stimulus;
a quantity of a drug, the drug being initially contained with the plurality of molecular-valves; and
a porous substrate including a surface area, the plurality of molecular-valves being disposed on the surface area of the porous substrate; and
exposing the molecular-valves of the drug delivery system to an exterior stimulus configured to selectively open the molecular-valves so as to release a drug.

20. A method as recited in claim 19, wherein the molecular-valves are activated so as to release the drug initially contained therein by one or more stimuli selected from the group consisting of photonic energy, electrical energy, a magnetic field, and a chemical concentration.

21. A method as recited in claim 19, wherein the molecular-valves are activated by exposure to a chemical concentration which results in a redox chemical reaction which alters the shape of the molecular-valve so as to permit release of the drug initially contained therein.
